(19) United States
(12) Patent Application Publication
(10) Pub. No.: US 2018/0221535 A1
(43) Pub. Date: Aug. 9, 2018

(54) SANITARY BARRIER FILMS

(71) Applicants: Ellis R. Burks, Moreland Hills, OH (US); Mark Deland Soucek, Akron, OH (US); Brittany Ann Pellegrene, North Canton, OH (US); Junyoung Seo, Akron, OH (US); Kevin Albert Cavicchi, Wadsworth, OH (US)

(72) Inventors: Ellis R. Burks, Moreland Hills, OH (US); Mark Deland Soucek, Akron, OH (US); Brittany Ann Pellegrene, North Canton, OH (US); Junyoung Seo, Akron, OH (US); Kevin Albert Cavicchi, Wadsworth, OH (US)

(21) Appl. No.: 15/888,913

(22) Filed: Feb. 5, 2018

Related U.S. Application Data
(60) Provisional application No. 62/454,233, filed on Feb. 3, 2017.
(60) Provisional application No. 62/624,923, filed on Feb. 1, 2018.

(51) Int. Cl. A61L 26/00; C08F 220/18; C08F 212/08; C08F 218/08

(52) U.S. Cl. CPC A61L 26/0014; C08F 220/18; C08F 2220/1858; C08F 218/08; C08F 212/08

(57) ABSTRACT

A polymer film is provided that adheres to the outer epidermal surface of human skin, for instance, and is suitable to be worn as a protective covering in the general nature of a glove, bandage, or wound closure, for instance. The film is durable and may be peeled off in more or less one piece without damaging or irritating the skin. The film is also discreet insofar as it is substantially clear and colorless, as well as thin. Thus, the film may go unnoticed by casual observers.

This application incorporates by reference, in their entireties, United States provisional patent application Nos. 62/454,233 filed on Feb. 3, 2017 and 62/624,923 filed on Feb. 1, 2018.

### I. BACKGROUND OF THE INVENTION

### A. Field of Invention

The present invention generally relates to the field of flexible polymer films formed on an outer surface of an epidermis.

### B. Description of the Related Art

There is a well-known need for products that protect individuals from contact with illness-causing bacteria and viruses. This is especially important with respect to the hands, since people are prone to unwittingly contaminating their hands and then touching food, rubbing their eyes, or engaging in some other activity that may infect them. One very well-known solution to contaminated hands is to simply wash with soap and water. However, soap and water are not always readily available, especially when one is on public transportation, for instance. Moreover, soap tends to dry the skin, so repeated use can be damaging. Antiseptic lotions are also known which contain compounds such as ethyl alcohol to kill bacteria. However, these wear off quickly and must be reapplied.

Compositions are known which may be applied to the skin for various purposes, e.g. wound care, and may form films of one type or another. However, all known compositions have significant problems that preclude them from functioning as an effective sanitary barrier. The following table refers to commercially available products including a short non-comprehensive summary of selected drawbacks. The products contained in this table are also shown in FIG. 1.

Table of Products

| Product | Principal Component(s) | Solvent | Drawbacks |
|---|---|---|---|
| Liquid Latex | Natural rubber latex | Water | Long dry time, |
| | | | allergen, |
| | | | color/opacity |
| Liquid Latex | Natural rubber latex | Water | Long dry time, |
| Fashions | | | allergen, |
| | | | color/opacity |
| Nexcare ® | Acrylate terpolymer | Organic | Difficult to peel, |
| | polyphenylmethylsiloxane | | skin irritant |
| New Skin ® | Nitrocellulose | Organic | Non-elastic film, |
| | | | skin irritant |
| Skin Shield ® | Nitrocellulose | Organic | Non-elastic film, |
| | | | skin irritant |
| Extra Skin ® | Polydimethly siloxane | Organic | No film |
| | | | formation, |
| | | | skin irritant |
| Vetbond ™ | n-butyl cyanoacrylate | None | Brittle film, |
| | | | not peelable |
| Skin Tite | Platinum-cure silicone | None | Tacky self- |
| | | | adhesive film, |
| | | | not peelable |

What is needed is a composition that can be applied in liquid form like a lotion, but quickly hardens forming a physical barrier like a glove. Some embodiments of the present invention may provide one or more benefits or advantages over the prior art.

### II. SUMMARY OF THE INVENTION

Some embodiments may relate to an optically clear and colorless copolymer film. The film has a tan(δ) between 0.75 and 1.3; a glass transition temperature between −10° C. and +37° C.; a modulus at 100% strain between 1 and 105 KPa; a peel adhesion strength between 10 and 35 N/m; a hardening time up to 15 minutes+/−3 minutes; and a thickness between 1 and 40 mils.

Embodiments may relate to copolymer films comprising either 2-ethylhexyl acrylate (EHA) or butyl acrylate (BA) at a weight percent between 2 and 40 wt %, or may include both EHA and BA at a weight percent independently selected from 1 to 30 wt %. Such embodiments may further include vinyl acetate (VAc) and methyl methacrylate (MMA) at a weight percent between 20 and 40 wt %. Embodiments may optionally include one of 2-hydroxyethyl methacrylate (HEMA), octyl carbamate ethyl methacrylate (OEM), acrylated methyl oleate (AMO), or methyl oleate at a weight percent up to 10 wt %. Embodiments may optionally include a rheological modifier effecting a viscosity between 300 and 4000 centipoise in a precursor emulsion. Moreover, the emulsion may have between 25 and 60 wt % solids content, and may produce an embodiment film between 1 and 40 mils thick. Suitable hardening times may be up to 15 minutes±3 minutes. Embodiment films may exhibit a tan(δ) between 0.75 and 1.3, and a peel adhesion strength between 10 and 35 N/m

The invention is not to be limited to the features summarized here. Other features, benefits, and advantages will become apparent to persons having ordinary skill in the art to which it pertains upon reading and understanding of the following detailed specification.

### III. BRIEF DESCRIPTION OF THE DRAWINGS

The invention may take physical form in certain chemical species, physical properties, and steps. Selected embodiments of the invention will be described in detail in this specification and illustrated in the accompanying drawings which form a part hereof, wherein like reference numerals indicate like structure, and wherein:

FIG. 1 contains images of prior art products that are applied to the skin for various purposes;

FIG. 2 is an illustration of a generalized emulsion polymerization process;

FIG. 3 is an illustration of a generalized latex film formation process;

FIG. 4 is an NMR spectrum of octyl carbamate ethyl methacrylate (OEM) in CDCl₃;

FIG. 5 is an NMR spectrum of acrylated methyl oleate (AMO) in CDCl₃;

FIG. 6 is a bar graph of T_{g} and MFFT of selected copolymer formulations;

FIG. 7 is a plot of stress versus strain of various formulations;

FIG. 8A is DMA strain sweep data for formulation MMA20;

FIG. 8B is DMA strain sweep data for formulation STY20;

FIG. 8C is DMA strain sweep data for formulation MO20;

FIG. 8D is DMA strain sweep data for formulation HEMA20;

FIG. 8E is DMA strain sweep data for formulation AMO20;

FIG. 8F is DMA strain sweep data for formulation OEM20;

FIG. 8G is DMA strain sweep data for formulation BA;

FIG. 8H is DMA strain sweep data for formulation EHA;

FIG. 8I is DMA strain sweep data for formulation BA-EHA; and

FIG. 9 is a bar graph showing tan(δ) values of selected formulations.

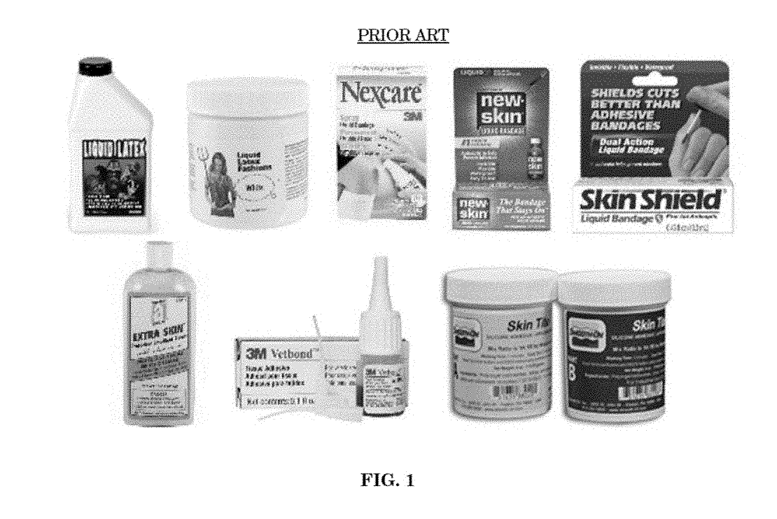

### IV. DETAILED DESCRIPTION OF THE INVENTION

As used herein the terms “embodiment”, “embodiments”, “some embodiments”, “other embodiments” and so on are not exclusive of one another. Except where there is an explicit statement to the contrary, all descriptions of the features and elements of the various embodiments disclosed herein may be combined in all operable combinations thereof.

Language used herein to describe process steps may include words such as “then” which suggest an order of operations; however, it is intended that the use of such terms is a matter of convenience and does not limit the process being described to a particular order of steps, except where it is stated that a particular order is contemplated.

Conjunctions and combinations of conjunctions (e.g. “and/or”) are used herein when reciting elements and characteristics of embodiments; however, unless specifically stated to the contrary or required by context, “and”, “or” and “and/or” are interchangeable and do not necessarily require every element of a list or only one element of a list to the exclusion of others.

Headings are used herein for the sake of convenience and are not intended to affect the meaning or scope of the specification and/or claims, but rather to make the specification more readily understandable. Headings should not be used to construe the meaning of any of the teachings set forth herein, or to limit the scope of the claims.

While the term room temperature is often taken to have a specific meaning, e.g. 25° C., as used herein this term is taken to be a range roughly between 20° C. and 25° C. inclusive. It will be understood by the skilled artisan, based on context, when the phrase room temperature is intended to indicate an instrument setting or measured quantity which may entail an ascertainable degree of precision and control, and when the phrase room temperature simply indicates ambient temperature. For instance, it is common in the art to indicate that chemical reactions are run at “room temperature”, “RT”, or often a number such as 25° C. is even given, but these indications are understood in the art to mean that the reaction process includes no temperature control at all. Thus, the reaction literally runs at whatever the ambient temperature happens to be plus or minus any thermal energy absorbed or released by the reaction. In contrast, where an instrument is intended to simulate room temperature, and a particular numerical value is provided, room temperature will be understood to indicate an appropriately well-known value.

Embodiments may comprise a polymeric sanitary barrier for the hands or other areas of the body. The barrier may be specially adapted to short-duration use after which it may be conveniently peeled off in more or less one piece and discarded. It may be particularly advantageous for embodiments to be discreet, fast-hardening, crack resistant, peelable, and non-irritating to the skin. Embodiments may be applied in the manner of a lotion, and may harden to a mechanically stable and pliable film on a timescale of seconds to minutes. Suitable films are substantially optically clear and colorless.

As used herein, the term hardening or hardened, as it relates to embodiments in film form or coating form, as in fast-hardening, means to solidify or gel from a liquid state or emulsion. This term is not intended to restrict embodiments to crosslinked polymers, un-crosslinked polymers, thermoset polymers, or thermoplastic polymers. Nor, is the term hardening or hardened intended to restrict embodiments to a process of formation, or even to a class of processes of formation such as drying or curing.

The term dry time is used herein to indicate a result of a specific test. Particularly, the test determines a time interval for a film to sufficiently harden so that it maintains its integrity while being subjected to certain physical conditions. The dry time determined according to these measurements is not intended to necessarily be equal to the time between the initial application of a precursor liquid, e.g. an emulsion, and the time when a user would perceive the film to be ready for use. In fact, it is contemplated that hardening may continue for a significant period after the user perceives the film as being ready to use. It is further contemplated that the user's perception would depend on factors such as whether the film feels sticky or wet to the point of being uncomfortable. Since such perceptions are inherently subjective, and thus may vary substantially from one person to another, embodiment films of the invention are characterized instead by dry times as defined above and discussed in more detail herein.

Embodiments may be a discreet coating or film. Suitably discreet materials may be characterized by being sufficiently thin, colorless, and clear to substantially blend in with the wearer's skin. It is contemplated that an operably hardened film of the invention is not necessarily invisible, but rather it tends to blend with the wearer's skin such that its presence is not immediately obvious, or that it does not call attention to itself and may very well go unnoticed by casual observers. Being substantially clear and colorless, embodiment films transmit the color of the wearer's skin. Embodiment films may also diminish, impede, or eliminate reflective shine from its surface. For instance, the surface may have sufficient roughness to reflect light diffusely enough to prevent reflective shine in many or most circumstances while not appearing white or cloudy, thus improving the embodiment's discreetness.

Film Thickness

The thickness of some embodiments in a hardened-film form may be on the order of the thickness of a typical examination glove having a few mils thickness. For illustration, and without limitation, an embodiments' thickness may be 3-8 mils. It is contemplated that greater thicknesses such as 9-25 mils or greater may be advantageous for certain embodiments and applications. Film thickness and discreetness are inversely related. Therefore, it is desirable to minimize thickness to maximize discreetness. However, the strength of a film formulation determines the lower limit of thickness because suitable films must be sufficiently strong to withstand the forces applied by a wearer to peel the film from the skin. Accordingly, lower Young's-modulus film embodiments must be thicker than higher Young's-modulus film embodiments. It is thus contemplated that a suitable film thickness, according to embodiments of the invention, is determined by a plurality of interrelated factors including, without limitation, tensile strength, peel strength, and film uniformity.

Film Uniformity

Another desirable and advantageous property of embodiments is a self-leveling property. Since embodiment films are applied similar to a lotion, e.g. by rubbing the composition between one's hands, it is not feasible to expect the user to carefully apply the composition so that it forms a uniform film. Rather, the embodiment may form a uniform film spontaneously, such as through the influence of surface tension and viscosity. The ordinarily skilled artisan will appreciate that suitable films of the invention need not be perfectly uniform in thickness. Rather, uniformity is acceptable when the thinnest part of the film is sufficiently strong to resist tearing during peel-off, while the thickest part of the film remains suitably discreet.

The degree of uniformity may be expressed as a standard deviation (a) in film thickness. Standard deviations within the scope of the invention may vary, and may depend in part on certain mechanical properties of a particular embodiment. For instance, a tougher or more cohesive film embodiment may tolerate a less uniform film thickness without failing and/or without fragmenting during peel-off. Conversely, a less-tough or less-cohesive film embodiment may tend to develop excessively weak regions that cannot tolerate forces applied during peel-off and/or within a predetermined range defined as normal wear.

Drying or Hardening Time

The terms drying time and hardening time are used interchangeably herein. Suitable hardening times of embodiments are on the order of seconds. Hardening is sufficiently slow to provide enough working time for a user to apply the embodiment, but it is sufficiently fast to avoid requiring the user to wait idly for the embodiment to harden. Some embodiments may be applied and function as a sanitary barrier almost immediately, even while the film is still hardening. Time ranges within the scope of the invention include without limitation between 1 and 10 seconds±0.5 seconds, 10 and 20 seconds±0.5 seconds, 20 and 30 seconds±0.5 seconds, 30 and 40 seconds±0.5 seconds, 40 and 50 seconds±0.5 seconds, and/or 50 and 60 seconds±0.5 seconds, or any combination thereof.

Some embodiments may be applied under conditions that permit longer hardening times without seeming objectionable to the user. For instance, a hardening time on the order of minutes to as much as 15 minutes±3 minutes may be suitable for certain applications such as bandages or wound closures on surfaces other than that of the palms of the hands. In general, longer hardening times become more tolerable to a user the less it interferes with the user's activities. Accordingly, an embodiment film deposited on the skin of a user's palms may require a faster hardening time than an embodiment applied elsewhere.

The person having ordinary skill in the art will appreciate that a number of methods, standards, and instruments are known for measuring drying times or hardening times. One such device is a ball and stylus instrument available from Gardco, which is used to test samples according to embodiments of the invention. This type of device includes a stylus comprising an arm and ball assembly mounted to a motor shaft. The stylus is thus driven in a circular arc at a constant speed over the surface of a drying or curing sample. The sample is deemed dry or cured when the stylus no longer marks the sample. Since this stylus moves at a constant speed for a known time across a known path length, the dry time can be accurately deduced from the point in the arc path where the marking ends. According to the nature of this measurement, the dry time or hardening time does not indicate that the sample loses no further moisture, or does not continue to coalesce, beyond the dry time. Indeed, drying may continue past the point when it is deemed dry by this method. The dry point according to this methodology simply means that the sample has reached a sufficient degree of hardening that stylus no longer marks the sample.

Suitable films within the scope of the present invention also resist cracking during the hardening process. Crack resistance according to some embodiments of the invention may include resistance to capillary forces that result from evaporation of a liquid phase from a drying latex emulsion or from a gel such as a hydrogel or alcogel. Crack resistance according to embodiments of the invention may also include resistance to applied forces such as shear forces and compressive forces. For example, embodiment films applied to the hands are at least capable of resisting fracture and cracking when the user engages in most ordinary activities such as gripping a railing, turning a door knob, opening a car door, or buttoning and unbuttoning clothes. Embodiments may also be sufficiently cohesive and tough to be peeled off the skin in more or less one piece. Suitable materials need not be peelable in exactly one piece, or without tearing, cracking, or fracture to any degree. Rather embodiments may be conveniently removed from the skin without expending undue time and effort.

Compositions and Formulations

It is contemplated that an embodiment may be applied in liquid-form, hardened, peeled off, and the liquid reapplied multiple times per day over an extended indefinite period without irritation to the skin. Accordingly, suitable compositions are non-irritating and hypoallergenic. Embodiments may optionally include compounds for soothing, moisturizing, or healing the skin such as, without limitation, aloe, essential oils, mineral oil, petrolatum, lanolin, dimethicone, hyaluronic acid, glycerin, and any of a wide variety of compounds known to have emollient, humectant, or healing properties.

Waterborne latexes provide certain advantages, one of which is in the number of different monomers that can be used to make a synthetic latex polymer. This allows the ordinarily skilled artisan to choose from a wide variety of commercially available inexpensive feedstocks to synthesize compounds for forming sanitary barrier films. Accordingly, mechanical and thermal properties of the film can be adjusted or tuned by varying the monomers used and their proportions relative to each other. For example, properties such as glass transition temperature, elasticity, peel strength, minimum film formation temperature (MFFT), and to a certain extent even dry time can be tuned in this way.

Embodiments of the invention may include synthetic waterborne latex-forming compounds. For example, and without limitation, embodiments may include copolymer latex formulations of one or more of vinyl acetate (VAc), 2-ethylhexyl acrylate (EHA), 2-hydroxyethyl methacrylate (HEMA), octyl carbamate ethyl methacrylate (OEM), acrylated methyl oleate (AMO), styrene (Sty), butyl acrylate (BA), and methyl methacrylate (MMA); one or more non-copolymerizing plasticizers including methyl or ethyl esters of a cis-olefinic acid having between 10 and 20 carbons, such as without limitation methyl oleate.

Latex formulations of the invention may include embodiments incorporating the foregoing monomers in a number of different combinations and amounts. For instance, and without limitation some embodiments may include either EHA or BA, while others may include both EHA and BA. Still other embodiments may include neither monomer. Accordingly, these two monomers may be present in amounts varying independently between 0 and 40 wt %. Embodiments may also include between 20 wt % and 40 wt % VAc, between 20 wt % and 40 wt % MMA, and another component selected from HEMA, AMO, OEM, and methyl oleate present at a weight percent above trace levels to an upper limit of 5 wt % to 10 wt %. In contrast to HEMA, AMO, and OEM methyl oleate may remain a discrete molecular species rather than copolymerize.

While the examples described in more detail herein include copolymers of the foregoing monomers, other copolymer compositions embodying the invention may be more broadly encompassed by the following structural formulae.

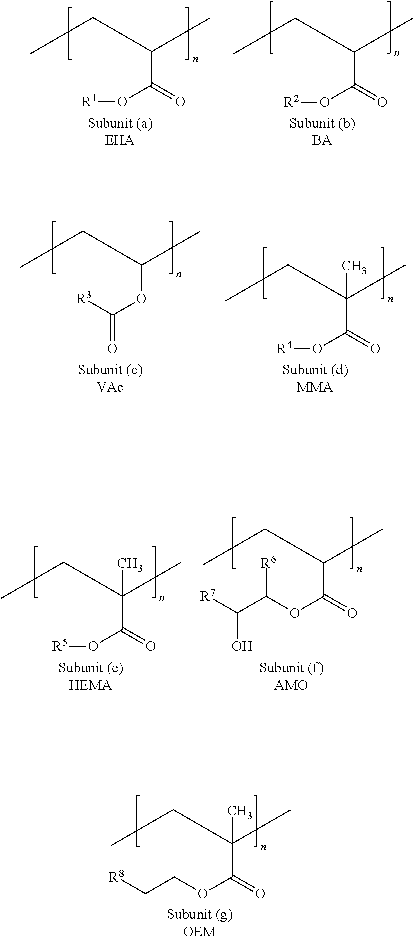

Copolymers of the invention may include either Subunit (a) or Subunit (b), or they may include both Subunit (a) and Subunit (b). Accordingly, Subunits (a) and (b) may be present at a percent by weight varying independently between 0 wt % and 40 wt %, wherein R¹ is a branching alkyl having 6 to 8 carbons and R² is an n-alkyl having 2 to 8 carbons. Subunit (c) may be present at a percent by weight between 20 wt % and 40 wt % wherein R³ is an alkyl having 1 to 3 carbons. Subunit (d) may be present at a percent by weight between 20 wt % and 40 wt % wherein R⁴ is an alkyl having 1 to 3 carbons. Additionally, embodiment formulations may include one of Subunits (e), (f), or (g) at a percent by weight above trace levels up to an upper limit of 5 wt % to 15 wt % wherein R⁵ is an n-alkyl alcohol having between 1 and 3 carbons, R⁶ is an n-alkyl methyl ester having between 6 and 10 carbons, R⁷ is an n-alkyl chain having between 6 and 12 carbons, and R⁸ is an n-alkyl carbamate having between 6 and 10 carbons. Instead of residues, (e), (f), or (g) formulations according to embodiments of the invention may include a cis-olefinic acid having between 10 and 20 carbons present at a weight percent above trace levels to an upper limit of 5 wt % to 15 wt %. The cis-olefinic acid having between 10 and 20 carbons may be, without limitation, methyl oleate.

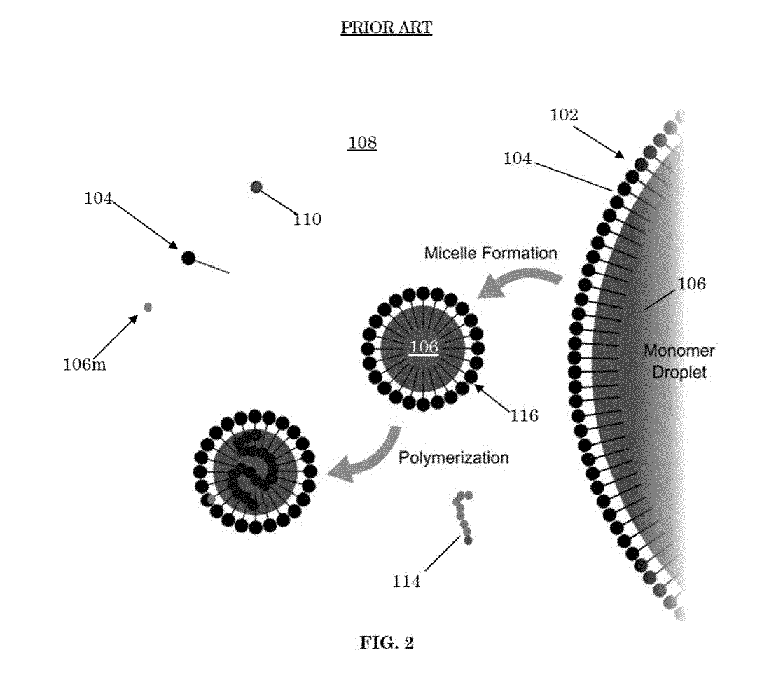

Referring now to the drawings wherein the showings are for purposes of illustrating embodiments of the invention only and not for purposes of limiting the same, FIG. 2 is a generalized depiction of an emulsion polymerization process that is typical in making waterborne latexes of the present invention. The components of an emulsion polymerization include water 108, monomer 106, an initiator 110, and a surfactant 104. A relatively large monomer droplet 102 is shown surrounded by surfactant molecules 104, which stabilize the hydrophobic droplet 102 in a water phase 108. Initiators 110 and excess surfactant molecules 104 are present in the water phase 108 as solvated molecular species. Monomer molecules 106m migrate from the monomer droplet 102 into the water phase 108 where they reactively interact with initiator molecules 110 thus beginning a polymerization reaction. As the polymer chain 114 grows, it becomes too hydrophobic to stably remain in the water phase 108. Accordingly, the polymer chain 114 enters a micelle 116 of surfactant molecules 104. The micelle also contains monomer 106 which enables the polymerization reaction to continue growing the polymer chain. This process continues until all the monomer 106 is depleted.

Emulsion polymerization products within the scope of the invention have particle sizes in the range of 80 to 100 nm, 100 to 120 nm, 120 to 140 nm, 140 to 160 nm, 160 to 180 nm, 180 to 200 nm or any operable combination thereof. Moreover, embodiment may comprise bimodal mixtures of two different particle sizes within suitable polydispersity limits as would be understood by the person having ordinary skill in the art.

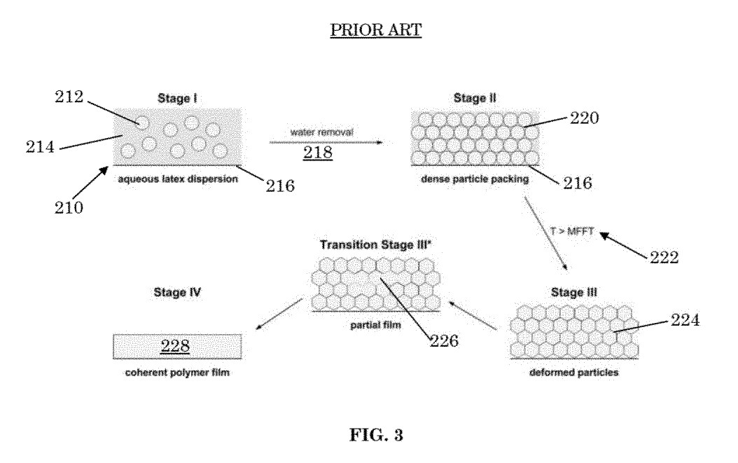

A generalized film formation process is illustrated in FIG. 3. An emulsion 210 of solid polymer 212 particles is shown dispersed in an aqueous continuous phase 214. The polymer particles 212 are stabilized in suspension by surfactant molecules (not shown). Film formation begins by evaporating the continuous phase 214 as shown in step 218. This causes the polymer particles 212 to compact 220 and to settle on surface 216. Provided sufficient thermal energy is available, the particles will also begin to coalesce. The minimum film formation temperature (MFFT) of a latex is the lowest temperature at which coalescence can occur. Below the MFFT, a continuous polymer film will not properly form. Rather, interfaces will remain between polymer particles. Above the MFFT 222 the polymer particles 212 will deform 224, filling the interstices between particles 212, and as densification continues the interfaces between particles breakdown 226 resulting in coalescence and ultimately in a continuous film 228. The ordinarily skilled artisan will appreciate that the MFFT is affected by a number of variables, among which chemical composition is fundamental. Accordingly, the combination of monomers used to make a polymer, and their relative proportions, weigh heavily in determining the MFFT of a polymer formulation.

Certain monomers impart particularly advantageous properties to copolymer latex formulations of the invention. Namely, these monomers include (i) vinyl acetate (VAc), (ii) 2-ethylhexyl acrylate (EHA), (iii) 2-hydroxyethyl methacrylate (HEMA), (iv) octyl carbamate ethyl methacrylate (OEM), and (v) acrylated methyl oleate (AMO), (vi) styrene (Sty), (vii) butyl acrylate (BA), and (viii) methyl methacrylate (MMA).

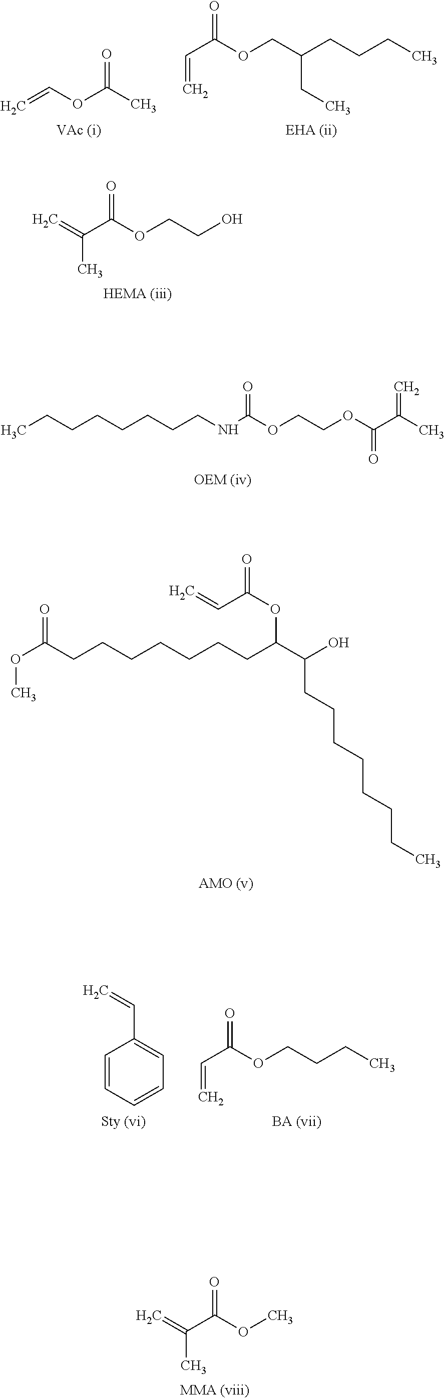

Vinyl acetate (VAc) tends to contribute to oxygen and water permeability. Since film embodiments of the invention may be applied directly to the skin, these permeability properties promote comfort. 2-Ethylhexyl acrylate (EHA) tends to promote adhesion of film embodiments to the skin. 2-Hydroxyethyl methacrylate (HEMA) tends to promote hydrogen bonding which cohesively strengthens film embodiments and further promotes adhesion to skin. Octyl carbamate ethyl methacrylate (OEM) tends to promote hydrogen bonding through the carbamate group, which again promotes adhesion and film strength of embodiments. The octyl chain of OEM tends to plasticize embodiment films. Lastly, the acrylated methyl oleate (AMO) provides plasticizing properties to embodiment films.

### Example OEM Monomer Synthesis

Octyl carbamate ethyl methacrylate (OEM) is prepared by a two-step synthesis process according to Reaction Scheme I. Ethylene carbonate (x) (88.06 g, 1 mol) is dissolved in 300 mL of dichloromethane in a 1-L three-neck flask. The flask is placed in an ice bath at 0° C. with a magnetic stirrer and N₂ atmosphere. Octyl amine (ix) (142.16 g, 1.1 mol) is then added dropwise to the solution. The reaction mixture is stirred for 24 h. The resulting solid, hydroxyoctylcarbamate (xi) is obtained after evaporation of dichloromethane under reduced pressure. Unreacted monomers are removed by dissolving the solid into water at 50° C. and pure hydroxyoctylcarbamate (xi) is obtained by filtration. The second step of the reaction begins with dissolving the hydroxyoctylcarbamate (xi) (108.65 g, 0.5 mol) in about 300 mL of dichloromethane in a 1-L three-neck flask. The flask is placed under a N₂ atmosphere in an ice water bath to keep the reaction at 0° C. while stirring. 4-(Dimethylamino) pyridine (DMAP) catalyst (610.9 mg, 5 mmol) and hydroquinone inhibitor (88.09 mg, 0.8 mmol) are added, followed by the dropwise addition of triethyl amine (TEA) (70.83 g, 0.7 mol), and then dropwise addition of methacrylic anhydride (xii) (92.5 g, 0.6 mol). The reaction is stirred under N₂ atmosphere at 0° C. for 24 h, yielding the OEM (iv) product.

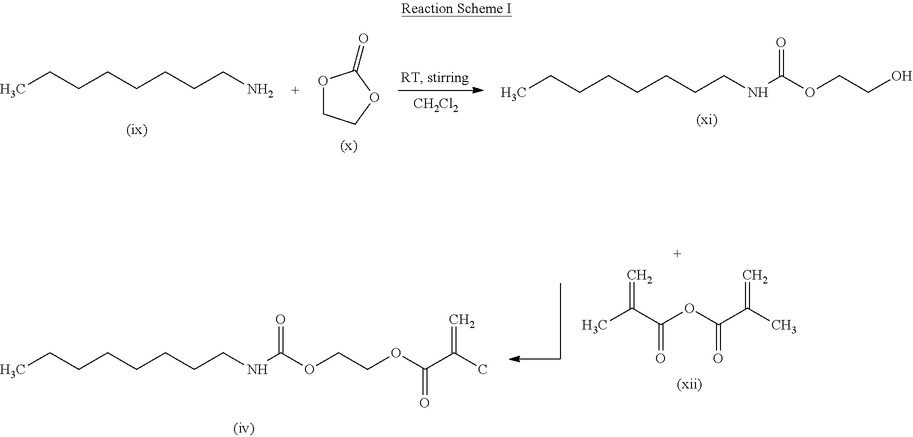

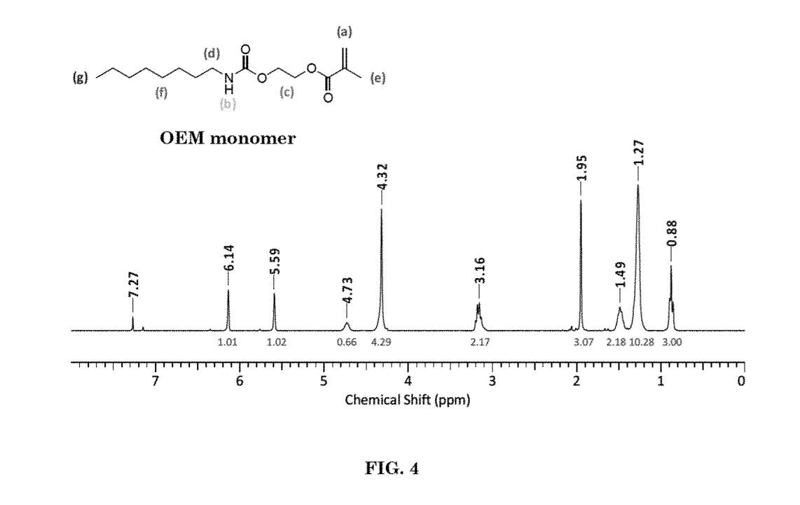

The OEM product (iv) is then extracted by addition of 200 mL of dichloromethane and washing with the following solutions: saturated brine (300 mL), 1 M hydrochloric acid solution (300 mL×3), saturated sodium bicarbonate solution (300 mL×3), and saturated brine (300 mL). The OEM is then dried over magnesium sulfate. The dichloromethane is then evaporated under reduced pressure. The OEM (iv) product is verifiable by ¹H-NMR according to the following example spectral analysis, the corresponding spectrum being illustrated in FIG. 4.

¹H-NMR (CDCl₃, 500 MHz, ppm): 0.88 (t, 3H, CH₃CH₂), 1.27 (m, 10H, CH₃(CH₂)5CH₂), 1.49 (m, 2H, CH₂CH₂NH), 1.95 (s, 3H, CH₃C═CH₂), 3.16 (m, 2H, —CH₂NH—), 4.32 (s, 4H, O(CH₂)2O), 4.73 (s, 1H, NH), 5.59 (s, 1H, CH₂═C), 6.14 (s, 1H, CH₂═C).

### Example Acrylated Methyl Oleate (AMO) Monomer Synthesis

The AMO monomer is prepared in a two-step process, according to Reaction Scheme II, starting with epoxidization of the double bond in methyl oleate (xiii) yielding structure (xix), and then acrylating by an esterification reaction to yield the AMO product (v). A round-bottom flask is charged with methyl oleate (100 g, 0.3373 mol). Formic acid (50.22 g, 1.09 mol) and hydrogen peroxide (78.38 g, 2.30 mol) are added dropwise. This solution is stirred vigorously in an ice bath for 16 h. Following the reaction, an ether extraction is performed. The resulting product is taken in 100 mL diethyl ether, and then washed with the following solutions: distilled water (100 mL), saturated sodium bicarbonate (100 mL, repeat until pH is neutral), and saturated brine (100 mL). The intermediate product, epoxidized methyl oleate (xix), is then dried over magnesium sulfate and the ether is evaporated under reduced pressure.

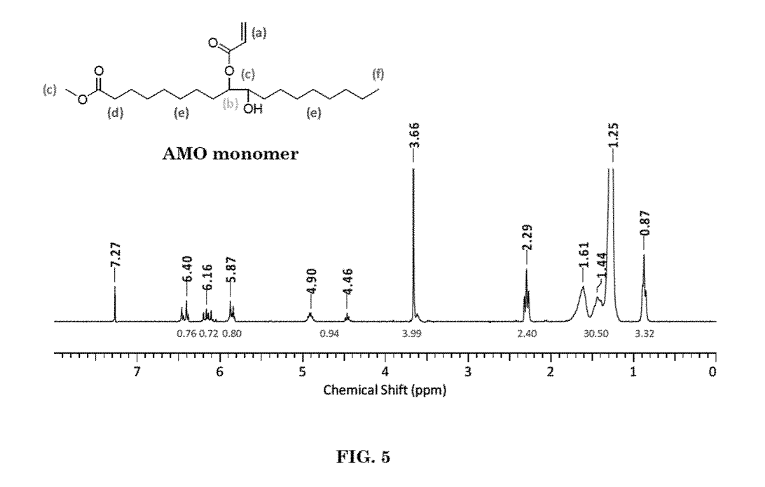

The resulting product is then acrylated by a catalyzed reaction with acrylic acid. In a round-bottom flask, equipped with a reflux condenser, the following are mixed: acrylic acid (28.96 g, 0.4019 mol), epoxidized methyl oleate (xix) (83.60 g, 0.2679 mol), hydroquinone inhibitor (0.2582 g, 0.0023 mol), and AMC-2 catalyst (0.8360 g). The components are stirred vigorously at 90° C. for 6 h. The catalyst used is a chromium (III) organometallic compound obtained from AMPAC Fine Chemicals, and catalyzes the reaction of epoxide and acid while tending to minimize side reactions such as homopolymerization. The structure of AMC-2 is proprietary, but it contains 50% trivalent organic chromium complexes and 50% phthalate esters. Following the reaction, the product is extracted by dissolving in 100 mL of diethyl ether, and washing with the following solutions: distilled water (100 mL), saturated sodium bicarbonate (100 mL, repeat until pH is neutral), and saturated brine (100 mL). The final product, epoxidized methyl oleate, is then dried over magnesium sulfate and the ether is evaporated under reduced pressure. The product is verifiable by ¹H-NMR according to the following example spectral analysis, the corresponding spectrum being illustrated in FIG. 5.

¹H-NMR (CDCl₃, 500 MHz, ppm): 0.87 (t, 3H, CH₃CH₂), 1.25-1.61 (m, 30H, CH₂), 2.29 (t, 2H, CH₂C═O), 3.66 (s, 3H, CH₃O), 4.46-4.90 (m, 1H, CHOC═O), 5.87-6.40 (m, 3H, CH═CH₂).

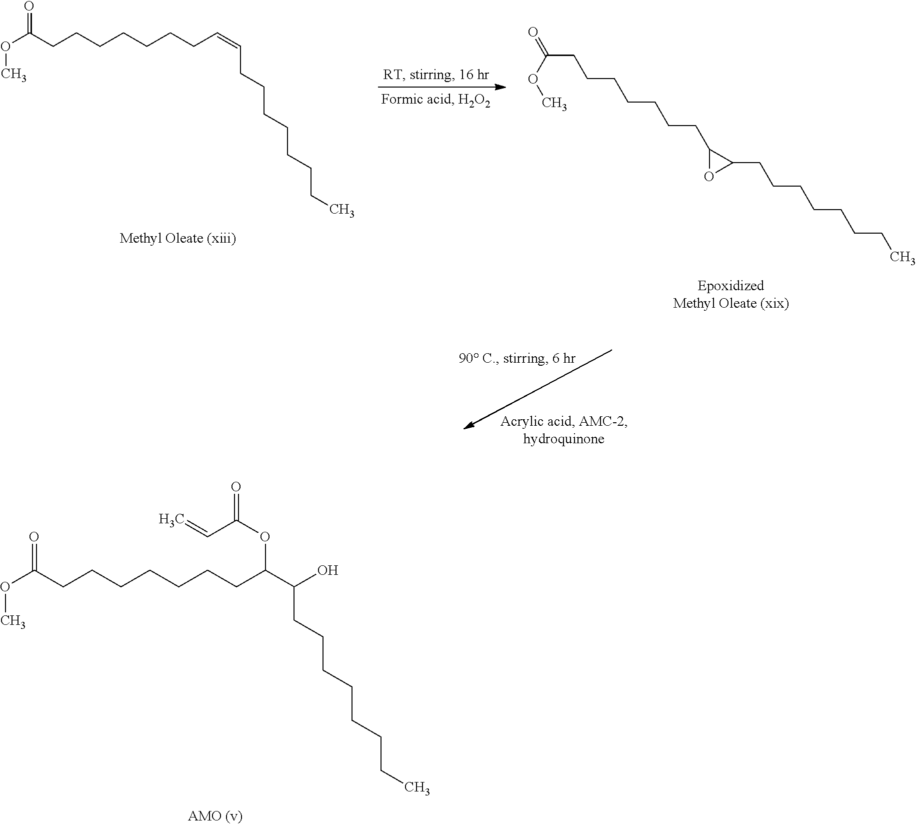 [SMILES: CCCCCCCC/C=C\CCCCCCCC(=O)OC ; CCCCCCCCC1OC1CCCCCCCC(=O)OC ; C=CC(=O)OC(CCCCCCCC(=O)OC)C(O)CCCCCCCC]

Seeding Polymer Reactions

The person having ordinary skill in the art will appreciate that a monodisperse or nearly monodisperse polymer product may be desirable for controlling mechanical and thermal properties. Moreover, embodiments may also benefit from bimodal mixtures of two different latex particle sizes to reduced drying time according to theory and methods known in the art.

In a broad sense polymer molecular weight depends on the interplay between the rate of particle nucleation and the rate of particle growth. Nucleation itself depends on the rate of radical formation, which can be highly variable. Accordingly, more reproducible molecular weights with a narrower polydispersity can be obtained by seeding the polymerization reaction.

### Example Seed Synthesis

A seed synthesis in accord with embodiments of the invention may be performed in a Optimax reactor, available from Mettler Toledo, equipped with a heating jacket, a mechanical stirrer, nitrogen inlet/outlet ports, and a reflux condenser. The initial charge to the reactor contains buffer, sodium bicarbonate (0.50 g), initiator, sodium dodecyl sulfate (0.33 g) and deionized water (50 g). The initial charge is stirred at 400 rpm and heated to 75° C. A monomer solution is then made by first adding sodium bicarbonate (0.03 g) and sodium dodecyl sulfate (1.0 g) to deionized water (26.67 g). This is stirred until a clear solution is formed, and then monomer (26.67 g) is added. An initiator solution is also made by dissolving ammonium persulfate (0.80 g) in deionized water (33.33 g). The initiator and monomer solutions are added dropwise over 2 h. The reaction is then stirred at 400 rpm and 75° C. for 2 h.

### Example Latex Synthesis

All latexes in accordance with embodiments of the invention described herein are synthesized by the same basic procedure while varying the amount of the following monomers: EHA, VAc, MMA, styrene, BA, MO, HEMA, AMO, and OEM. The latexes may be made in an Optimax reactor, equipped with a heating jacket, a mechanical stirrer, nitrogen inlet/outlet, and a reflux condenser. A seed solution (5.33 g), in accordance with processes described herein, is charged to the reactor. The reactor is set to 55° C. with stirring at 300 rpm under a N₂ atmosphere. A monomer solution is made by adding surfactants, Triton X-100 (0.64 g) and sodium dodecyl sulfate (0.16 g), and sodium bicarbonate buffer (0.03 g) to deionized water (26.67 g). This solution is stirred until all solids are dissolved, and then the monomers (26.67 g) are added. An initiator solution is also made by adding ammonium persulfate (0.80 g) to deionized water (33.33 g). The initiator and monomer solutions are added to the reactor dropwise over 2 h. The reaction is stirred at 300 rpm and 55° C. for 4 h.

The person having ordinary skill in the art will understand that it may be advantageous to make certain adjustments to the continuous phase of a latex prepared according to the foregoing example. For instance, the amount of continuous phase may be reduced to decrease drying time allowing for more rapid film formation. Alternatively, or additionally, drying time may be reduced by adding a low-boiling liquid such as an alcohol, e.g. methanol, ethanol, iso-propanol, or n-propanol, which may have beneficial azeotropic effects further decreasing dry time. The person having ordinary skill in the art will also appreciate that such adjustments are limited by double layer effects bearing on the stability of the emulsion.

Tables 2A and 2B show selected latex formulations, comprising embodiments of the invention, which are synthesized according to the methods described herein. The formulations of Table 2A exhibit relatively high glass transition temperatures, while those of Table 2B are significantly lower. Furthermore, the formulations of Table 2A are applied in an emulsion form having a solids content of about 20%, whereas those of Table 2B have a solids content of about 40%. Accordingly, the films produced by Table 2B formulations are significantly thicker than those of Table 2A. Suitable solid contents within the scope of the invention can be between about 15 and 60 wt %.

TABLE 2A
Higher T_{g} Copolymers

| Sample | % EHA | % VAc | % MMA | % Sty | % MO | % HEMA | % AMO | % OEM | T_{g} (° C.) |
|---|---|---|---|---|---|---|---|---|---|
| MMA0 | 33.4 | 33.3 | 33.3 | — | — | — | — | — | 2.2 |
| MMA20 | 30.2 | 23.3 | 46.5 | — | — | — | — | — | 21.5 |
| MMA30 | 23.9 | 23.7 | 52.4 | — | — | — | — | — | 30.8 |
| MMA40 | 18.7 | 29.1 | 52.1 | — | — | — | — | — | 43.3 |
| STY0 | 32.3 | 32.2 | — | 35.4 | — | — | — | — | 1.6 |
| STY10 | 29.3 | 22.6 | — | 47.5 | — | — | — | — | 13.2 |
| STY20 | 23.0 | 33.2 | — | 43.7 | — | — | — | — | 19.8 |
| STY30 | 20.0 | 24.4 | — | 55.6 | — | — | — | — | 33.6 |
| STY40 | 19.2 | 19.3 | — | 61.4 | — | — | — | — | 41.8 |
| MO20 | 27.9 | 29.7 | 42.4 | — | 3.0 | — | — | — | 21.2 |
| HEMA10 | 25.0 | 35.0 | 35.0 | — | — | 5.0 | — | — | 13.0 |
| HEMA20 | 25.9 | 24.9 | 43.4 | — | — | 5.0 | — | — | 16.9 |
| AMO20 | 20.9 | 21.0 | 52.9 | — | — | — | 5.3 | — | 19.3 |
| OEM20 | 20.1 | 37.5 | 37.3 | — | — | — | — | 5.1 | 17.1 |

TABLE 2B
Lower T_{g} Copolymers

| Sample | % EHA | % VA_{c} | % MMA | % BA | % RM | % MO | % HEMA | % AMO | % OEM | T_{g} (° C.) |
|---|---|---|---|---|---|---|---|---|---|---|
| EHA | 33 | 33 | 33 | — | — | — | — | — | — | −1 |
| BA-EHA | 18 | 36 | 29 | 18 | — | — | — | — | — | 5 |
| BA | — | 40 | 20 | 40 | — | — | — | — | — | −5 |
| BA-RM | — | 40 | 20 | 40 | 6 | — | — | — | — | −5 |
| BA-MO | — | 36 | 29 | 36 | — | 3 | — | — | — | 4 |
| BA- | — | 34 | 27 | 34 | — | — | 5 | — | — | 13 |
| HEMA | | | | | | | | | | |
| BA-AMO | — | 34 | 27 | 34 | — | — | — | 5 | — | 8 |
| BA-OEM | — | 34 | 27 | 34 | — | — | — | — | 5 | 0 |

Formulation BA-RM of Table 2B includes 6 wt % of a rheological modifier, Rheolate® 420, having the effect of increasing the viscosity of the emulsion and film thereby producing a more uniform coating. Rheolate® 420 is commercially available from Elementis Specialties of East Windsor, N.J. The chemical structure and composition of Rheolate® 420 is proprietary, however, it is known to be an alkali-swellable acrylic emulsion. The person having ordinary skill in the art will readily appreciate that other rheological modifiers are available and may be substituted according to well know methods and principles to obtain a similar effect in producing suitable emulsion viscosities. Suitable emulsion viscosities can range from 300 to 4000 centipoise (cps). More specifically, suitable viscosities can be between 300 and 400 cps, 400 and 500 cps, 500 and 750 cps, 750 and 1000 cps, 1000 and 1250 cps, 1250 and 1500 cps, 1500 and 1750 cps, 1750 and 2000 cps, 2000 and 2250 cps, 2250 and 2500 cps, 2500 and 2750 cps, 2750 and 3000 cps, 3000 and 3250 cps, 3250 and 3500 cps, 3500 and 3750 cps, 3750 and 4000 cps, or any operable combination thereof.

Thermal Properties

The glass transition temperature of the latex film embodiments described herein may be analyzed with a TA Instruments Q2000 Differential Scanning calorimeter (DSC). Minimum film formation temperature (MFFT) may be measured with a Rhopoint Instruments MFFT 60.

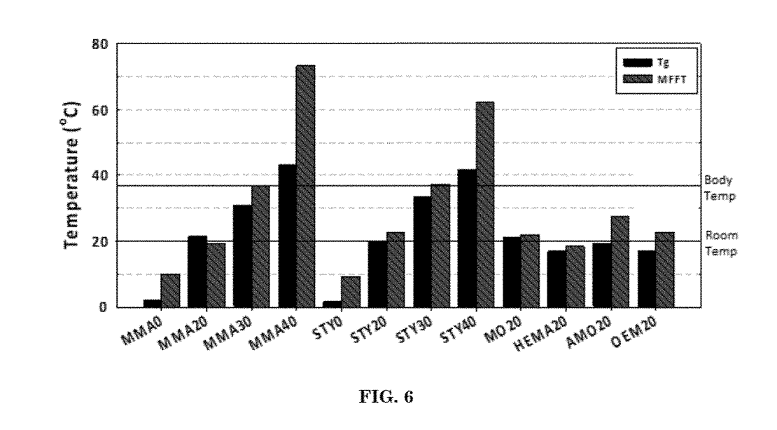

T_{g} and MFFT data are compiled and displayed graphically in FIG. 6 for the formulations of Table 2A. This graph principally shows a comparison of formulations having increasing levels of one of two high-T_{g} monomers, namely methyl methacrylate (MMA) or styrene (STY). Specifically, the MMA formulations (MMAO, MMA20, MMA30, and MMA40) comprise various combinations of EHA, VAc, and MMA, while the STY formulations (STYO, STY20, STY30, and STY40) comprise various combinations of EHA, VAc, and styrene. The relative amounts of the high-T_{g} monomer are chosen to vary the T_{g} of each formulation from about −5° C.+/−5° C. to about 40° C.+/−5° C. in ten-degree steps. Accordingly, the formulations have glass transition points encompassing room temperature (20° C.) and body temperature (37° C.).

Four embodiments in Table 2A comprise various combinations of EHA, VAc, and MMA plus a plasticizing component, and have a T_{g} around 20° C. Namely, these formulations include MO20, HEMA20, AMO20, and OEM20. A plot of the T_{g} and MFFT of each formulation is shown in FIG. 6, illustrating glass transition temperatures around 20° C. and MFFTs below room temperature for each formulation. The plasticizing components of these four formulations are about 5 wt. % of methyl oleate (MO), 2-hydroxyethyl methacrylate (HEMA), acrylated methyl oleate (AMO), or octyl carbamate ethyl methacrylate (OEM).

The MO20, HEMA20, AMO20, and OEM20 formulations exhibit properties that are generally similar to that of the MMA20 films. AMO20 is more brittle than the others, which is surprising due to its similarity to the methyl oleate plasticizer used in MO20. However, AMO20 contains a larger amount of MMA due to the low T_{g} of AMO, which may explain to the AMO20 film's relative brittleness. Among the MO20, HEMA20, AMO20, and OEM20 formulations, it is found that films with a T_{g} between 10-20° C. exhibit more favorable combination of properties. Importantly, this range applies to these particular embodiment formulations and not embodiments overall. Suitable glass transition temperatures can be found as low as −10° C. and as high as body temperature, i.e. about 37° C.

Tensile Strength

Films are dried at 37° C. overnight and then aged at room temperature for seven days prior to testing. Films are advantageously suitable for making measurements with a tensiometer. For instance, suitable films may have a length and width around 30 mm and 13 mm, respectively, and a thickness around 0.3-0.4 mm. Tensile properties of the films may be analyzed with an Instron 5567 tensiometer at room temperature (20° C.), and an Instron 4204 tensiometer at body temperature (37° C.).

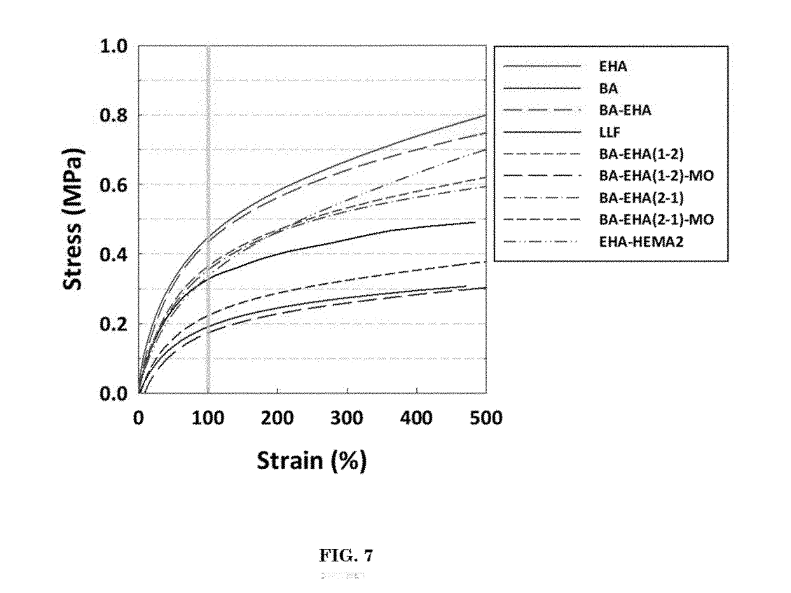

As shown in Tables 3A and 3B below, MMA20 exhibits a relatively high tensile strength of 8 MPa±1 MPa at room temperature and it exhibits the highest tensile strength at body temperature with a tensile strength of 1.8 MPa±0.5 MPa. In addition to tensile strength, elasticity is another important factor in formulations embodying the present invention. Selected tensile test data is shown in FIG. 7, indicating a modulus at 100% strain (a/k/a Mod 100 or Modulus 100) between about 2 and 4.5 KPa. Suitable ranges for embodiments of the invention also include 1 to 10 KPa, 1 to 2 KPa, 2 to 3 KPa, 3 to 4 KPa, 4 to 5 KPa, 5 to 6 KPa, 6 to 7 KPa, 7 to 8 KPa, 8 to 9 KPa, 9 to 10 KPa, or any operable combination thereof.

TABLE 3A

| | Tensile | | | |
|---|---|---|---|---|
| | Strength | Tensile | Modulus | T_{g} |
| Sample −20° C. | (MPₐ) | Strain_{(%)} | (MPₐ) | (° C.) |
| MMA0 | 2.2 ± 0.7 | 4.4 ± 0.8 | 0.9 ± 0.3 | 2.2 |
| MMA20 | 8 ± 1 | 2 ± 1 | 240 ± 30 | 21.5 |
| STY10 | 5.2 ± 0.7 | 5.7 ± 0.4 | 19 ± 5 | 13.2 |
| STY20* | 9.8 | 0.05 | 684 | 19.8 |
| MO20 | 2.2 ± 0.3 | 0.5 ± 0.3 | 33 ± 2 | 21.2 |
| HEMA10 | 6.1 ± 0.9 | 2.9 ± 0.2 | 2.4 ± 0.5 | 13.0 |
| OEM20** | 5 ± 2 | 0.04 ± 0.03 | 300 ± 100 | 17.1 |

TABLE 3B

| | Tensile | | | |
|---|---|---|---|---|
| | Strength | Tensile | Modulus | T_{g} |
| Sample −37° C. | (MPₐ) | Strain_{(%)} | (MPₐ) | (° C.) |
| MMA0*** | 0.49 ± 0.09 | 6.7 ± 0.4 | 0.3 ± 0.1 | 2.2 |
| MMA20 | 1.8 ± 0.5 | 4.6 ± 0.7 | 0.54 ± 0.06 | 21.5 |
| STY10*** | 0.36 ± 0.03 | 8 ± 1 | 0.25 ± 0.08 | 13.2 |
| STY20*** | 0.8 ± 0.5 | 8 ± 1 | 0.15 ± 0.04 | 19.8 |
| MO20 | 0.36 ± 0.08 | 1.4 ± 0.2 | 1.3 ± 0.3 | 21.2 |
| HEMA10 | 0.9 ± 0.3 | 4 ± 2 | 0.4 ± 0.1 | 13.0 |
| OEM20** | 0.9 ± 0.2 | 2.4 ± 0.7 | 70 ± 10 | 17.1 |

Elasticity

Elasticity may be evaluated by dynamic mechanical analysis (DMA) instrument, such as a TA Q800, using a strain sweep method. Suitable films for making DMA measurements have a length and width around 10 mm and 8 mm, respectively, and a thickness around 0.3-0.4 mm. The person having ordinary skill in the art will understand that a strain sweep is a well-known viscoelastic measurement wherein a stress is cyclically applied to a material at a constant frequency while steadily increasing the strain amplitude. Strain sweep test results are presented in terms of the ratio loss modulus (E″) over of storage modulus (E′), which is referred to as the tangent of δ, or simply tan(δ). The loss modulus relates to the energy dissipation of an embodiment film, or its viscous component, and the storage modulus relates to the elasticity of the film. Thus, the ratio tan(δ) indicates the degree to which a material is more viscous or more elastic. In general, if the loss modulus of a film is higher than its storage modulus (tan(δ)>1), it will tend to plastically deform under stress. Conversely, if storage modulus is higher than loss modulus (tan(δ)<1), then the film will tend to elastically recover to its original shape. Suitable values of tan(δ) range between about 0.75 and 1.3. Other suitable ranges include 0.75 to 0.8, 0.8 to 0.9, 0.9 to 1.0, 1.0 to 1.1, 1.1 to 1.2, 1.2 to 1.3, or any operable combination thereof.

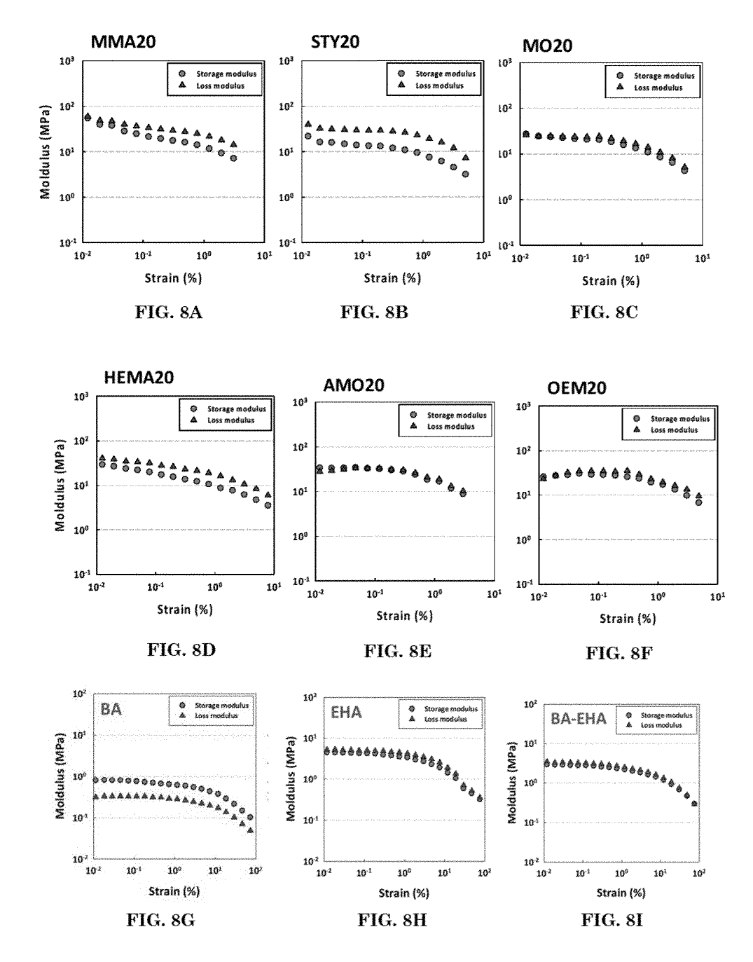

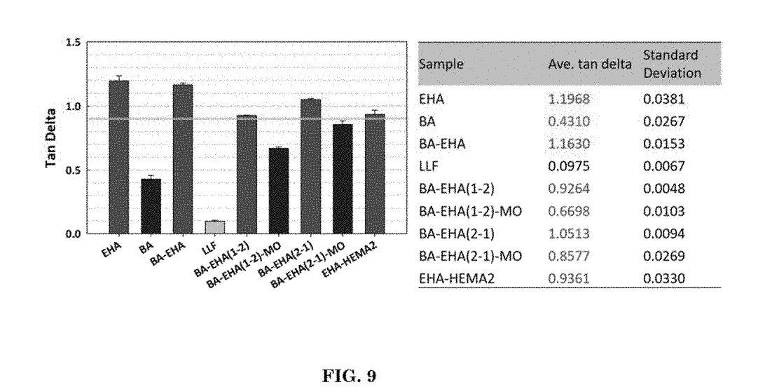

Selected strain sweep test results are illustrated in FIGS. 8A through 8F for six different embodiment formulations of Table 2A, namely MMA20, STY20, MO20, HEMA20, AMO20, and OEM20 respectively. In each instance, loss modulus exceeds storage modulus. Moreover, the DMA data suggests that embodiment formulations may include a plasticizer, and a lower wt % of the high T_{g} component to bring T_{g} below 20° C. In the illustrated embodiments described herein the high T_{g} component tends to be methyl methacrylate, however, embodiments are not limited to using MMA as the high-T_{g} component.

Each of the formulations shown in FIGS. 8A through 8F include EHA as a component. In contrast, the formulations of Table 2B illustrate embodiments where EHA is replaced either partially or fully with BA. As shown in FIGS. 8H and 8I, embodiment formulations having EHA only or equal amounts EHA and BA both exhibit a larger loss modulus than storage modulus. In contrast, the embodiment of FIG. 8G contains 40 wt % BA, 40 wt % VAc, and 20 wt % MMA. As shown in FIG. 8G in the absence of EHA storage modulus is significantly greater than loss modulus, indicating that BA produces a much more elastic embodiment than EHA.

This result is especially surprising because BA and EHA have similar molecular structures. Notably, EHA has a relatively high entanglement molecular weight due to chain branching, which tend to increase the loss modulus of an embodiment. More specifically, the results described herein suggest that EHA tends to result in films having a loss modulus that is higher than its storage modulus. In contrast, BA has a lower entanglement molecular weight, and tends to produce films with a higher storage modulus.

Peel Adhesion Testing

Porcine skin is a suitable model system for studying peel adhesion. Skins can be obtained from a local butcher shop, and the hair is shaved from the surface. The skin is heated to 37° C. to mimic human skin at body temperature. A latex sample is applied to the skin and allowed to dry. Testing is performed by peeling the latex from the skin surface with a suitable testing apparatus as described herein. A second test is performed by bending and stretching the skin to determine whether the latex remains adhered under realistic usage conditions.

Regarding adhesion testing, a porcine skin sample is prepared by gluing it to a metal substrate so that it can be held upright for the peel testing. The peel testing can be performed at 180° angle to the substrate using the Instron 4204 tensiometer. The prepared porcine skin sample is held by the stationary clamp, and the film is attached to the top clamp, which is moved at a crosshead speed of 50 mm/min. The peel strengths of the films are measured to gather information on the adhesive properties of various embodiment formulations. Suitable formulations have peel strengths between 10 and 35 N/m. Other suitable peel strength ranges include without limitation, 10 to 15 N/m, 15 to 20 N/m, 20 to 25 N/m, 25 to 30 N/m, 30 to 35 N/m, or any operable combination thereof.

Although much of the discussion herein focuses on latex formulations, the person having ordinary skill in the art will appreciate that any material having suitable physical properties, as defined and claimed herein, is within the scope of the invention. More specifically, suitable physical properties include, without limitation, a tan(δ) between 0.75 and 1.3 at 37° C. in a linear viscoelastic strain regime of the copolymer; a glass transition temperature between −10° C. and +37° C.; a modulus at 100% strain between 1 and 10 KPa; a peel adhesion strength between 10 and 35 N/m; a hardening time up to 15 minutes+/−3 minutes; and a thickness between 1 and 40 mils. Although not required for all embodiments, some embodiments may be colorless and/or optically clear. However, other embodiments may be colored, and may even have coloring or dyes added without departing from the scope of the invention. Moreover, embodiments may scatter visible light and thus may be cloudy or opaque rather than optically clear.

It will be further apparent to those ordinarily skilled in the art that the above methods and apparatuses may be changed or modified without departing from the general scope of the invention. The invention is intended to include all such modifications and alterations insofar as they come within the scope of the appended claims or the equivalents thereof.

Having thus described the invention, it is now claimed:

We claim:

1. A copolymer film, comprising:
either 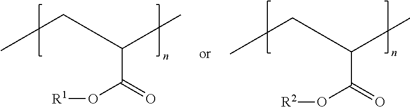
present at a percent by weight between 2 wt % to 40 wt %; 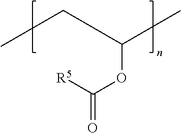
present at a percent by weight between 20 wt % to 40 wt %; 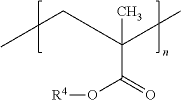
present at a percent by weight between 20 wt % to 40 wt %;
wherein R¹ is a branching alkyl having 6 to 8 carbons, R² is an n-alkyl having 2 to 8 carbons, R³ is an alkyl having 1 to 3 carbons, and R⁴ is an alkyl having 1 to 3 carbons;
the copolymer film having a peel strength from bare skin at 37° C. between 10 N/m and 35 N/m; and
the copolymer having a tan(δ) between 0.75 and 1.3 at 37° C. in a linear viscoelastic strain regime of the copolymer.

2. The copolymer film of claim 1, wherein 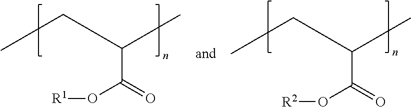
are both present at a percent by weight between 1 wt % to 30 wt %.

3. The copolymer film of claim 2, further comprising an additional component selected from one of 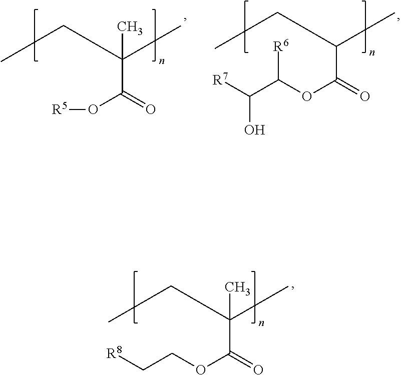
a methyl or ethyl ester of a cis-olefinic acid having between 10 and 20 carbons, at a percent by weight above trace up to an upper limit of 15 wt %, wherein R⁵ is 2-hydoxy ethyl, R⁶ is an n-alkyl methyl ester having between 7 and 8 carbons, R⁷ is an n-alkyl chain having between 6 and 12 carbons, and R⁸ is an n-alkyl carbamate having between 7 and 9 carbons.

4. The copolymer film of claim 3, further comprising a rheological modifier present at a percent by weight above trace up to 12 wt % and effecting a viscosity of a precursor emulsion of the copolymer film between 300 centipoise and 4000 centipoise.

5. The film of claim 4, wherein the emulsion comprises between 25 wt % and 60 wt % solids content.

6. The film of claim 5 having a thickness between 1 mils and 40 mils.

7. The film of claim 1, having a hardening time up to 15 minutes±3 minutes.

8. The film of claim 7, having a hardening time up to 10 seconds±3 seconds.

9. The film of claim 1, having a tan(δ) between 0.90 and 1.20 at 37° C. in a linear viscoelastic strain regime of the copolymer.

10. The film of claim 1, having a peel adhesion strength between 15 and 25 N/m.

11. A copolymer film, comprising:
either 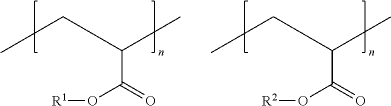
or present at a percent by weight between 2 wt % to 40 wt %; 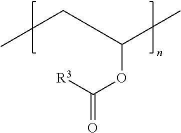
present at a percent by weight between 20 wt % to 40 wt %; 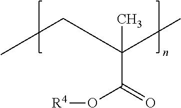
present at a percent by weight between 20 wt % to 40 wt %;
wherein R¹ is 2-ethyl hexyl, R² is n-butyl, R³ is an alkyl having 1 to 3 carbons, and R⁴ is an alkyl having 1 to 3 carbons;
the copolymer film having a peel strength from bare skin at 37° C. between 10 N/m and 35 N/m; and
the copolymer having a tan(δ) between 0.75 and 1.3 at 37° C. in a linear viscoelastic strain regime of the copolymer.

12. The copolymer film of claim 11, wherein both 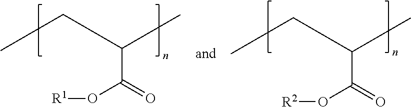
are present at a percent by weight between 2 wt % to 18 wt %.

13. The copolymer film of claim 12, further comprising one of 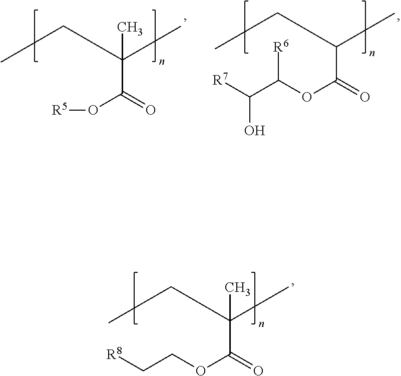
or methyl oleate
at a percent by weight above trace up to an upper limit of 15 wt %, wherein R⁵ is an n-alkyl alcohol having between 1 and 3 carbons, R⁶ is an n-alkyl methyl ester having between 6 and 10 carbons, R⁷ is an n-alkyl chain having between 6 and 12 carbons, and R⁸ is an n-alkyl carbamate having between 6 and 10 carbons.

14. The copolymer film of claim 13, further comprising a rheological modifier present at a percent by weight above trace up to 9 wt % and effecting a viscosity of a precursor emulsion of the copolymer film between 500 centipoise and 3500 centipoise.

15. The film of claim 14, wherein the emulsion comprises between 30 wt % and 50 wt % solids content.

16. The film of claim 15 having a thickness between 3 mil and 25 mils.

17. The film of claim 11, having a hardening time up to 1 minute±20 seconds.

18. The film of claim 110, having a tan(6) between 0.9 and 1.0 at 37° C. in a linear viscoelastic strain regime of the copolymer.

19. The film of claim 11, having a peel adhesion strength between 15 and 25 N/m.

20. A copolymer film having:
a tan(δ) between 0.75 and 1.3 at 37° C. in a linear viscoelastic strain regime of the copolymer;
a glass transition temperature between −10° C. and +37° C.;
a modulus at 100% strain between 1 and 10 KPa;
a peel adhesion strength between 10 and 35 N/m;
a hardening time up to 15 minutes+/−3 minutes; and
a thickness between 1 and 40 mils.
